(19) United States
(12) Patent Application Publication
(10) Pub. No.: US 2012/0088959 A1
(43) Pub. Date: Apr. 12, 2012

(54) ORGAN PACKING DEVICE HAVING TRANSFORMABLE SUPPORT MEMBERS

(76) Inventor: Anthony P. Deasey, Severna Park, MD (US)

(21) Appl. No.: 13/166,622

(22) Filed: Jun. 22, 2011

Related U.S. Application Data
(60) Provisional application No. 61/392,462, filed on Oct. 12, 2010.

(51) Int. Cl. A61F 2/04

(52) U.S. Cl. 600/37

(57) ABSTRACT

An elastomeric device for packing the organs of a subject. The device comprises a central portion and one or more flaps collectively manually positionable within the subject to retain the organs of the subject in an operational, displaced position and to provide a surgical operational space; and at least one transformable support member disposed in at least one of the central portion and the flaps configured to transform from a first substantially compliant configuration to a second substantially rigid configuration.

### CROSS-REFERENCE TO RELATED APPLICATIONS

The contents of U.S. Provisional Patent Application No. 61/392,462 filed on Oct. 12, 2010, U.S. Provisional Patent Application 61/125,219 filed on Apr. 23, 2008, and PCT/US2009/002495 filed on Apr. 22, 2009, are hereby incorporated herein by reference in their entirety.

### BACKGROUND

1. Field of the Invention

The present invention relates generally to organ packing, and more particularly, to a organ packing device having transformable support members.

2. Related Art

Abdominal and pelvic procedures generally require displacement and retention of organs or other organs to create a space that allows the surgeon to perform the procedure. This step of displacement and retention of organs is referred to herein as organ packing.

The current laparotomy packing procedure used in the operating room today is time-consuming relative to the overall priorities of events in a surgery. The surgeon first uses his hands to displace the organs away from the surgical site. Intra-abdominal sponges and towels are then used to pack the organs out of the way. Finally, abdominal retractors are fitted over the dressings with gentle traction to hold the cotton sponges in place.

This conventional organ packing causes several issues during surgery. For instance, organ packing may take up to ten minutes, and, because the organs have a tendency to protrude from the dressing into the surgical space, the organ packing must be repeated frequently during extended surgical procedures, taking additional time. Additionally, the cotton sponges used to pack the organs are made of loose cotton fibers that are abrasive to the intestines and can adhere to the organs, and remain within the subject even after removal of the sponges. These fibers can promote peritoneal inflammation, a major cause of post-operative adhesion formation. Furthermore, the sponges tend to dry out over the course of the surgical procedure, becoming abrasive and adhesive to the organs themselves, further contributing to the formation of adhesions, a leading cause of post-operative morbidity. Finally, because multiple sponges are used, there is a danger that one or more sponges will be forgotten in the abdominal cavity.

For laparoscopic surgery, the current method is to use gravity to encourage the organs to move out of the surgical field (Trandelenburg method). Additionally, laparoscopic forceps may be used to move sections of the organ out of the surgical field. These methods are time consuming, require an additional assistant, and are difficult to get a stable packing of the organs.

### SUMMARY

According to one aspect of the present invention, there is provided an elastomeric device for packing the organs of a subject. The device comprises a central portion and one or more flaps collectively manually positionable within the subject to retain the organs of the subject in an operational, displaced position and to provide a surgical operational space; and at least one transformable support member disposed in at least one of the central portion and the flaps configured to transform from a first substantially compliant configuration to a second substantially rigid configuration.

According to another aspect of the present invention, there is provided a method of packing organs of a subject with a device including a central portion and one or more flaps, wherein the device comprises at least one transformable support member disposed in at least one of the central portion and the flaps configured to transform from a first substantially compliant configuration to a second substantially rigid configuration. The method comprises accessing an interior of an abdominal cavity of the subject; repositioning the organs to provide a surgical space in the abdominal cavity; positioning the device having the at least one transformable support member in the first configuration abutting the organs; and transforming the at least one transformable support member to the second configuration to provide a barrier between the organs and the surgical space.

According to another aspect of the present invention, there is a elastomeric device for packing the organs of a subject for a laparoscopic procedure. The device is collapsible to allow it to be inserted into a small incision or a trocar. After the device is inside the abdominal cavity, the transformable support structures are transformed to a second substantially rigid configuration and then used to pack the organs. After the surgery, the device is transformed back to a substantially compliant configuration and removed via the small incision or trocar.

### BRIEF DESCRIPTION OF THE DRAWINGS

Embodiments of the present invention are described below with reference to the attached drawings, in which:

FIG. 1 is a front view of a organ packing device having transformable support members, in accordance with embodiments of the present invention;

FIG. 2A is a front view of a organ packing device having transformable support members, in accordance with embodiments of the present invention;

FIG. 2B is a front view of the organ packing device of FIG. 2A;

FIG. 3 is a front view of a organ packing device having transformable support members, in accordance with embodiments of the present invention;

FIG. 4 is a front view of a organ packing device having transformable support members, in accordance with embodiments of the present invention;

FIG. 5 is a front view of a organ packing device having transformable support members, in accordance with embodiments of the present invention;

FIG. 6 is a front view of a organ packing device having transformable support members, in accordance with embodiments of the present invention;

FIG. 7 is a front view of a organ packing device having transformable support members, in accordance with embodiments of the present invention;

FIG. 8A is a perspective view of a transformable support member, in accordance with embodiments of the present invention;

FIG. 8B is a perspective view of the transformable support member of FIG. 8A;

FIG. 9 is a flowchart of a method for using a organ packing device having transformable support members, in accordance with embodiments of the present invention; and

FIG. 10 is a perspective view of a subject in which a organ packing device in accordance with embodiments of the present invention may be implemented.

### DETAILED DESCRIPTION

Aspects of the present invention are generally directed to device for packing or retaining the bowels or other organs of a subject during a laparotomy or laparoscopic surgical procedure. Such a device is referred to herein as a organ packing device. The organ packing device is formed from an elastomeric material and includes one or more support members adapted to be transformed from a first substantially compliant configuration to a second substantially rigid configuration. As described in detail below, these transformable support members may have a number of different structures and/or arrangements.

The use of a organ packing device in accordance with embodiments of the instant invention provides advantages over conventional sponges and towels not only in ease of use, but in improved patient outcomes. Specifically, the use of the organ packing device provides for a reduction in adhesion formation as a result of organ packing as compared to organ packing performed with sponges. Adhesions are due at least in part to fibers from sponges that are abrasive to the bowels and organs and that remain in the abdominal cavity after the removal of sponges at the end of the surgery. As the organ packing device of the present invention includes no exposed fibers, none can be left behind, eliminating at least one substantial cause of adhesions. The use of the organ packing device also decreases organ packing time, thereby decreasing the total surgical time. The overall surgical time reduction will depend on, in part, the number of times the organ would need to be re-packed during the surgery. Therefore, in some embodiments the organ packing devices allow for a reduction in operating room time, a reduction in anesthesia time, and a reduction in post-operative morbidity associated with the use of surgical sponges used in current procedures.

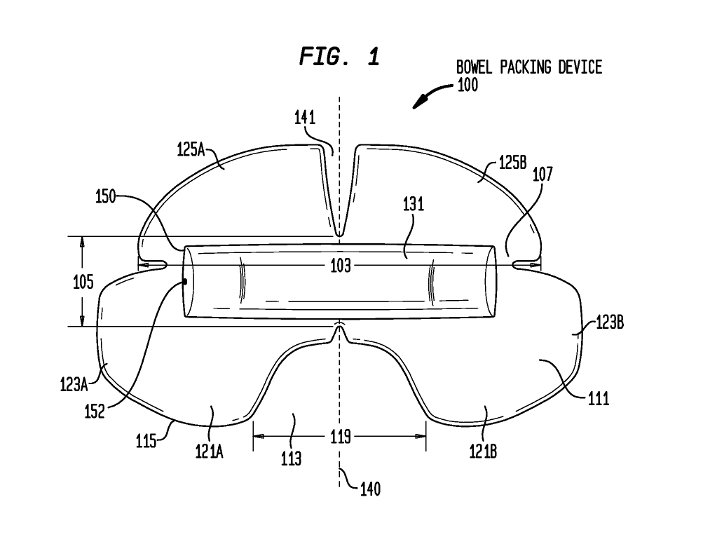

FIG. 1 is a front view of a bowel packing device 100 in accordance with embodiments of the present invention. As shown, bowel packing device 100 includes an essentially rectangular central portion 107 having a width 103 and a height 105. Device 100 further comprises first and second top flaps 125 separated by a notch 141, and first and second bottom flaps 121 separated by a notch 113. Additionally, device 100 further comprises two side flaps 123, Central portion 107 and flaps 121, 123, and 125, collectively form an essentially elliptical shape. More specifically, device 100 has an essentially elliptically-shape, that is generally symmetrical about a minor axis 140 of the device.

Notch 113 in FIG. 1 is a bell-shaped cut with in the bottom edge 115 of the device, and is provided to accommodate the ventral medial part of the body in the sagittal plane, and designed to conform to, and provide space for, the spinal cord towards the ventral wall of the abdominal cavity).

In the embodiments of FIG. 1, device 100 is formed from an elastomeric or polymeric compound, and device 100 further comprises a transformable support member 150. In this embodiment, transformable support member 150 is disposed in central region 107, and is configured to have a first substantially compliant configuration and a second substantially rigid or stiff configuration. When support member 150 is in the compliant configuration, central region 107 is flexible and may be compressed or otherwise manipulated by the surgeon. However, when support member 150 is in the rigid configuration, central region 107 is substantially stiff.

As described in greater detail below, a transformable support member in accordance with embodiments of the present invention may have a number of different arrangements and structures. In the embodiments of FIG. 1, support member 150 comprises an enclosed region 131 disposed in central region 107. Enclosed region 131 comprises, in this illustrative embodiment, a tubular shaped member formed within device 100. That is, the walls of enclosed region are formed from the body of device 100. In certain embodiments, the portions of device 100 forming the walls of enclosed region 131 are reinforced with an additional material, such as embedded fibers, or have thickness that is greater than other regions of the device.

Enclosed region 131 is configured to receive a pressurized fluid therein via an access port 152 that may comprise, for example, a valve. When support member 150 is in the compliant configuration, enclosed region 131 contains little or none of the pressurized fluid. However, when the support member 150 is in the rigid configuration, enclosed region 131 is substantially filled with the pressurized fluid. In certain embodiments, the pressurized fluid is a pressurized liquid, while in other embodiments the pressurized fluid is a pressurized gas.

As previously noted, during a organ packing procedure, a surgeon displaces the subject's organs to create a space that allows the surgeon to perform the procedure. Device 100 is used to retain the organs in this displaced position, thereby providing a barrier that maintains the surgical space. More specifically, a first surface of device 100 abuts the subject's organ. In certain embodiments of the present invention, transformable support member 150 provides sufficiently rigidity to the device such that the device may retain the organs in the displaced position without the need for additional surgical instructions. In such embodiments, device 100 is referred to herein as a self-retaining organ packing device.

In other embodiments, surgical instruments, such as one or more retractor blades, are used to retain device 100 in its operable position. Specifically, the retractor blades interface with a second surface 111 that opposes the first surface.

As used herein, organs generically include organ, intestine, and other abdominal organs that would need to be displaced in the abdominal cavity to allow for abdominal surgery. The standard retractor blade setup uses two blades that interface with the lateral sides of the body. Additionally, upon insertion of device 100 into a subject, side flaps 123 contact the lateral sides of the abdominal cavity and top flaps 125 contact the ventral side of the abdominal cavity. Side flaps 123 serve to aid in containing organs that may protrude around the sides of the device in the abdominal cavity. The purpose of top flaps 123 is to help secure the organs on the ventral side of the subject. In other words, device 100 is dimensioned to cover the organs of the subject when operationally positioned within the abdomen of the subject.

Bowel packing device 100 is appropriately sized for organ packing of a subject. That is, the device is dimensioned to allow for insertion into the abdominal cavity of the subject. For example, in adult humans, the size of the abdominal cavity is about 3.9 to 5.8 inches in the transverse plane at the height of the base of the ribs and about 7.6 to 11.3 inches in the coronal plane at the height of the base of the ribs. An appropriately sized device for organ packing in a mammal having such dimensions is about 5.2 to about 7.5 inches overall height (from ventral to dorsal sides of the abdominal cavity upon placement) and about 8.7 to about 12.5 inches in overall width (from lateral side to lateral side of the abdominal cavity upon placement). However, it would be appreciated that device 100 may have different sizes and shapes, depending on, for example, the insertion technique, surgical procedure, subject, etc. In certain embodiments, portion 107 has a width 103 that is approximately 7.82 inches, and height 105 that is approximately 3.63 inches. In such embodiments, notch 112 has a height 117 of approximately 2.28 inches and a base width 119 of approximately 4.00 inches. Further details of the shape and of body of a organ packing device in accordance with the embodiments of FIG. 1 are described in International Patent Application No. PCT/US2009/002495, filed on Apr. 22, 2009, the contents of which are hereby incorporated by reference herein. Specifically, bowel packing device 100 has substantially the same shape and size as provided for the organ packing device described in PCT/US2009/002495.

It would be appreciated that the shape, size, location of notches, etc., of body 102 of FIG. 1 is merely illustrative, and different embodiments are within the scope of the present invention. For example, in certain embodiments, device 100 may having a thicknesses that varies throughout the body. In one specific such embodiment, the portion of device 100 at an intersection of the major axis and the minor axis is greater than the thickness of the body at the perimeter. In another embodiment, device 100 may include radial notches of essentially any shape that are independently selected. Notch shapes include, but are not limited to V-shaped, U-shaped, and bell-shaped. Further details of variations for the shape, size, location of notches, etc., of device 100 may be as described in PCT/US2009/002495, the contents of which were previously incorporated by reference herein.

In the embodiments of FIG. 1, device 100 is formed from an elastomeric or polymeric compound such as a silicon polymer. As used herein, “elastomeric compound” is understood as an elastic compound having an appropriate flexibility/rigidity, tear resistance, and sterilization resistance for use in the devices of the invention. Elastomeric compounds for use for manufacture of the device of the invention are sufficiently flexible to prevent damage from occurring to tissues or organs by contact with the device when in a non-compressed state. Elastomeric compound as used herein typically refers to an elastomeric polymer. The monomers that link to form the polymer are typically made from of carbon, hydrogen, oxygen and/or silicon. Examples of elastomeric polymers include Liquid Silicone Rubbers (LSR) and Silicone Encapsulants. In a specific embodiment of the invention, the elastomeric polymer is a “silicone polymer”. A “silicone polymer” is understood as any silicone-based polymeric material that has the appropriate flexibility/rigidity, tear resistance, and sterilization resistance for use in the devices of the invention. In a further embodiment, the silicon polymer is optically clear. Elastomeric compounds for use in the device of the invention include, but are not limited to silicone, liquid silicone rubber (LSR), polydimethylsiloxane (PDMS), styrene butadiene rubber, styrene butadiene styrene (SBS) rubber, nitrile rubber, and polychloroprene (Neoprene), Polyvinyl Chloride (PVC), Polyethylene Terephthalate (PET), Polyurethane. In one embodiment, silicon polymer is polydimethylsiloxane (PDMS) a silicon-based organic polymer. PDMS is optically clear, and is generally considered to be inert, non-toxic and non-flammable. In some embodiments, the material for the device is of sufficient flexibility to permit folding, compressing, or roiling of the device to allow for insertion through a retracted incision as small as 10 cm in diameter, while being of sufficient rigidity to expand after folding, compression, or rolling, and retain the organs for the duration of a surgical procedure when used in conjunction with retractor blades.

In an exemplary embodiment, the main body of the device includes an inner core of Sylgard® 184 (Dow Corning) polydimethylsiloxane polymer between 8 and 14 mm in thickness, to provide rigidity to the main body, encased in a layer of Sylgard® 186 to confer improved tear-resistance and durability to the barrier. Flaps are made of a tear resistant silicon polymer, with sufficient flexibility to allow for adjustment of the flaps in the abdominal cavity, while providing sufficient rigidity to retain the barrier in place. Exemplary peripheral flap materials include Sylgard® 186 between 2 and 8 mm in thickness, projecting from the main body at angles of between 20 and 60 degrees, and decreasing in thickness with distance from the main body.

When using more than one elastomeric compound for manufacture of the device, the compounds can be used together in any manner. For example, a polymer with the desired rigidity can be coated with a polymer having greater smoothness. The device may be composed of one polymer, and the flaps can be composed of one or more other polymers to provide varying amounts of rigidity to the central portion and the flaps.

Further, in an embodiment, at least some portions of the device are made of a clear material which allows the organs to be visually monitored throughout the procedure, an advantage not allowed by the sponges used in current procedures. Further, the use of an elastomeric material provides for retention of both moisture and warmth in the abdominal cavity as compared to packing methods using surgical sponges.

Embodiments of the barrier (including the collapsible barrier) may be made, at least in part, from thermoplastic elastomers, such as by way of example, styrenic block copolymers, polyolefin blends, elastomeric TPU, thermoplastic copolyester, and thermoplastic polyamides, polysulfide rubber, and/or thermoplastic vulcanizates. Still further, thermoset elastomers, including polyisoprene, may be used to make at least some portions of the barrier, Saturated rubbers may also be used, such as, for example, EPM and EPDM, Epichlorohydrin rubber, polyacrylic rubber, fluorosilicone rubber, fluoroelastomers, perfluoroelastomers, polyether block amides, chlorosulfonated polyethylene, ethylene-vinyl acetate. Non-elastomeric polymers may also be used to make the barrier, including PTFE, PU, PET, LDPE, Cross-linked PE, HDPE, PE, Polypropylene, PEEK, PVC, polycarbonate, Polystyrene, and/or PEI. Composite materials may also be used, which may include the above-mentioned polymers and materials combined with reinforcing fibers, fillers, woven materials, polymer foam inserts, etc.

Polymers with relatively low Tg/softening points that would deform with steam sterilization may be used to manufacture the collapsible barrier. An embodiment of the present invention includes features/the use of materials that reduce the likelihood that the barrier may be reused, thus reducing the spread of disease.

As previously noted, in certain embodiments device 100 is formed from a material having a desired level of tear resistance. Tear resistance is the resistance of a material to initial tearing while tear strength represents the force required to tear a pre-slit material. For use in some embodiments, an un-slit material needs to have no visible tears develop upon application of 100 N of shear force. The amount of shear force required to tear pre-slit material may also be determined to identify potential failure modes of the barrier. In order to determine if Sylgard® 184 and or Sylgard® 186 may be able to withstand expected shear forces applied by the retractor blades on the body of the barrier, both tear resistance and tear strength of the material may be determined. Sylgard® 184 and 186 may be compared to each other to determine the most tear-resistant material. Force thresholds may be determined from measurements made in a simulated abdominal cavity.

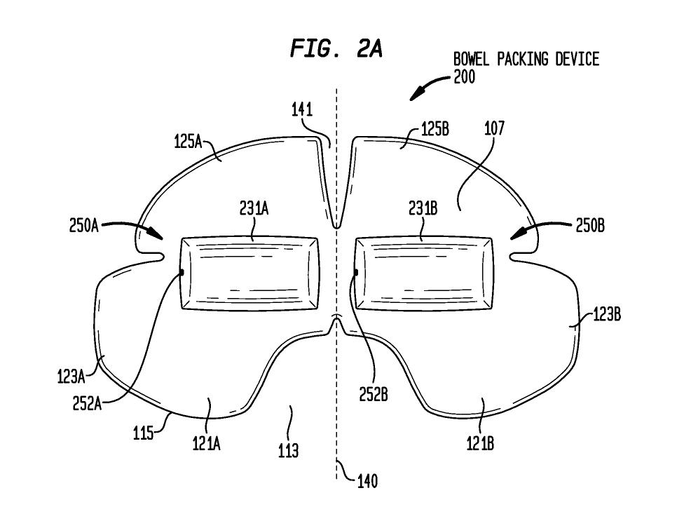

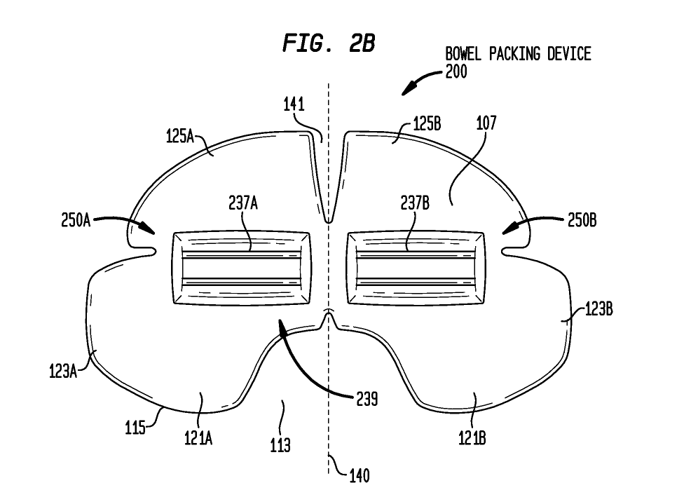

FIG. 2A is a front view of a organ packing device 200 in accordance with further embodiments of the present invention. Device 200 is similar to device 100 of FIG. 1 and includes a central portion 107 and flaps 121, 123 and 125. Bowel packing device 200 further comprises two transformable support members 250 disposed in central portion 107 of the device. Similar to support member 150 of FIG. 1, support members 250 are each configured to have a first substantially compliant configuration and a second substantially rigid or stiff configuration. When support members 250 are in the compliant configuration, central region 107 is flexible and may be compressed or otherwise manipulated by the surgeon. However, when support members 250 are in the rigid configuration, central region 107 is substantially rigid relative to the remainder of the device and/or the subject's tissue.

As described in greater detail below, a transformable support member in accordance with embodiments of the present invention may have a number of different arrangements and structures. In the embodiments of FIG. 2A, support members 250 each comprise an enclosed region 231 disposed in central region 107. Each enclosed region 131 comprises, in this illustrative embodiment, a tubular shaped member formed within device 200. That is, the walls of enclosed region are formed from the body of device 200. In certain embodiments, the portions of device 200 forming the walls of enclosed regions 231 are reinforced with an additional material, such as embedded fibers, or have thickness that is greater than other regions of the device.

In this embodiment, enclosed regions 231 contain a transformable material that transforms or transitions between fluid and solid states in response to application/removal thereto of thermal energy and/or pressure. Such transformable material may take a number of different forms. For example, in certain embodiments of FIG. 2A, transformable material is a wax having a melt pointing that is slightly above a subject's body temperature. As such, when the temperature of wax is raised above the subject's body temperature, the wax turns to a liquid and support members 250 are substantially flexible and conformable. However, as the wax cools below its melting point, the wax becomes a solid and support members 250 are substantially rigid relative to the remainder of the device and/or the subject's tissue.

As used herein, slightly above a subject's body temperature refers to temperatures that are above the body temperature, but that sufficiently low such that, if the device is at such temperature, the device will not damage the subject's tissue if inserted into the subject. In one specific example, such a temperature is less than 15 degrees Fahrenheit above the subject's body temperature. Exemplary waxes that may be used in embodiments of the present invention include, for example . . . a high oil content paraffin or soybean was . . . .

FIG. 2A is described above with reference to the use of a transformable material in the form of a wax having a melting point that is slightly above a subject's body temperature. It would be appreciated that other transformable materials may be used in embodiments of the present invention.

The wax in FIG. 2A is heated through the application of thermal energy to the wax. Thermal energy may be applied to the wax using a number of different methods. In one embodiment, the thermal energy is applied using thermal radiation from, for example, a heat lamp, or the like, or placing the device in an autoclave set at a relatively low temperature or limiting the temporal exposure of the barrier placed in an autoclave at a relatively high temperature. In other embodiments, the thermal energy is applied through the use of convention and/or conduction heat transfer. For example, the device might be placed in a warm-water or warm fluid bath, the device may be placed in an oven, an element emitting relatively high amounts of thermal energy may be placed against the barrier, etc. In still other embodiments, the device may be rubbed to introduce thermal energy via friction. In further embodiments, the thermal energy may be applied through delivery of an electrical current or upon exposure to non-thermal radiation of a given frequency (e.g., light). In some embodiments, the thermal energy is applied via microwaves of a microwave oven, in one such exemplary embodiment, the device includes a water-tilled reservoir adjacent the thermally-responsive material. Upon exposure of the water to microwaves, the temperature of the water increases, thereby heating the thermally-responsive material. These different methods of application of thermal energy are merely illustrative, and any means that will permit transfer of thermal energy to the device to decrease the stiffness of the thermally-responsive material may be used in embodiments of the present invention.

FIG. 2B is a front view of device of 200 of FIG. 2A in which the device includes a heating system 239 for delivery of electrical current to the device to heat the wax. In this illustrative embodiment, heating system 239 includes a plurality of electrical wires 237 that positioned adjacent to the wax. Wires 237 may be embedded in the wax, in the elastomeric material forming device 200, or on the surface of the device. Wires 237 are electrically connectable to a power source that provides current to the wires. Therefore, to facilitate insertion into a subject, current is passed, through wires 237, the heat from the wires increases the temperature of the wax above its melting point. While the wax is in the melted, liquid state, device 200 is inserted into the subject. In certain embodiments, when it is desired to remove device 200 from the subject, the wax may be again heated using system 239 so that support members 250 again become conformable.

Heating system 239 of FIG. 2B is merely illustrative, and it should be appreciated that other heating systems may be implemented in embodiments of the present invention. For example, in one alternative embodiment, the heating system includes a fluid circuit embedded in, or disposed on, the device. The circuit comprises inlet and outlet ports connected by fluid passages that pass a heated fluid, through to near the wax.

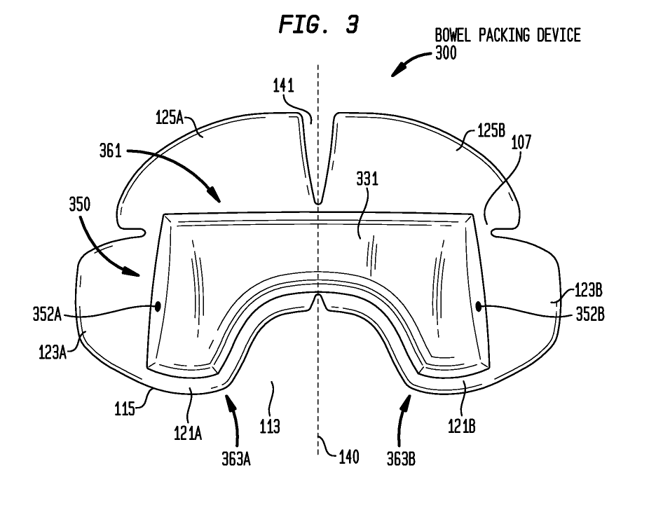

FIG. 3 is a front view of a bowel packing device 300 in accordance with further embodiments of the present invention. Device 300 is similar to device 100 of FIG. 1 and includes a central portion 107 and flaps 121, 123 and 125. Bowel packing device 300 further comprises a transformable support member 350 disposed in central portion 107 and flaps 121. Similar to support member 150 of FIG. 1, support member 350 is configured to have a first substantially compliant configuration and a second substantially rigid or stiff configuration. When support member 350 is in the compliant configuration, central region 107 and flaps 121 are flexible and may be compressed or otherwise manipulated by the surgeon. However, when support member 350 is in the rigid configuration, central region 107 and flaps 121 are substantially rigid relative to the remainder of the device and/or the subject's tissue.

As described elsewhere herein, a transformable support member in accordance with embodiments of the present invention may have a number of different arrangements and structures. In the embodiments of FIG. 3, support member 350 comprises an enclosed region 331 having a rectangular shape region 361 disposed in central portion 107, and two projections 363 that extend from region 361 into flaps 121. In the illustrative embodiment of FIG. 3, projections 363 have an area there between that is generally the same shape as notch 113. Enclosed region 331 is formed within device 300. That is, the walls of enclosed region are formed from the body of device 300. In certain embodiments, the portions of device 300 forming the walls of enclosed region 331 are reinforced with an additional material, such as embedded fibers, or have thickness that is greater than other regions of the device.

In the embodiment of FIG. 3, enclosed region 331 includes a transformable material 333 in the form of a wax. As noted above, the wax transforms or transitions between fluid and solid states in response to application/removal thereto of thermal energy. Specifically, the wax has a melt pointing that is slightly above a subject's body temperature. As such, when the temperature of the wax raised above the subject's body temperature, the wax turns to a liquid and support member 350 is substantially flexible and conformable. However, as the wax cools below its melting point, the wax becomes a solid and support member 350 is substantially rigid relative to the remainder of the device and/or the subject's tissue.

In the embodiments of FIG. 2 described above, the wax is positioned in enclosed regions 231 during manufacture of the device and is referred to as being permanently positioned in enclosed regions. In contrast, in the embodiments of FIG. 3, the wax is introduced into enclosed regions before or during a surgical procedure.

As shown in FIG. 3, support member 350 includes ports 352 that allow a surgeon to introduce a liquid wax into the enclosed region. As such, in the first, flexible configuration of support member 350, enclosed region 331 does not include the wax. However, in the second, rigid configuration of support member 350, the liquid wax inserted into enclosed region 331 and the wax cools to at or below the subject's body temperature.

It would be appreciated that the shapes and locations for region 361 and projections 363 identified above with reference to FIG. 3 are merely illustrative and do not limit embodiments of the present invention. For example, in other embodiments, projections 363 may be rectangular elements that extend from region 361. In still other embodiments, region 361 may have a circular, oval or other shapes. In further embodiments, projections 363 may also, or instead, extend into flaps 123 and/or 125.

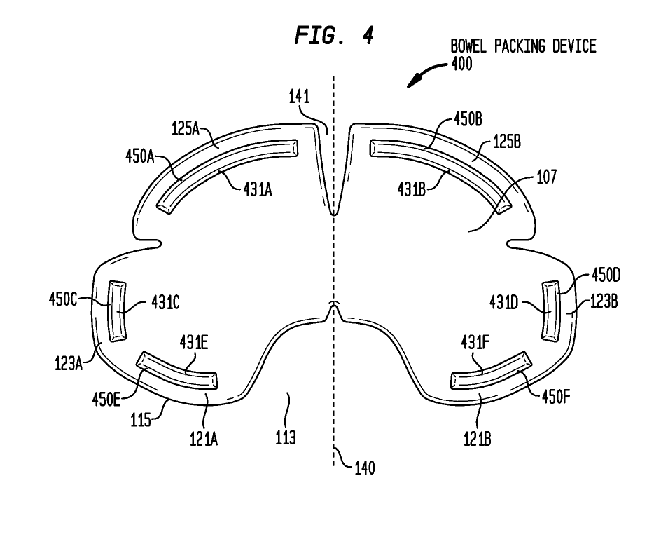

FIG. 4 is a front view of another bowel packing device 400 in accordance with embodiments of the present invention. Device 400 is similar to device 100 of FIG. 1, and includes a central portion 107 and flaps 121, 123 and 125. Bowel packing device 400 further comprises a plurality of transformable support members 450 disposed in flaps 121, 123 and 125 of the device.

Similar to support member 150 of FIG. 1, support members 450 are configured to have a first substantially compliant configuration and a second substantially rigid or stiff configuration. When support members 450 are in the compliant configuration, flaps 121, 123 and 125 are flexible and may be compressed or otherwise manipulated by the surgeon. However, when support members 450 are in the rigid configuration, flaps 121, 123 and 125 are substantially rigid relative to the remainder of the device and/or the subject's tissue.

As described elsewhere herein, a transformable support member in accordance with embodiments of the present invention may have a number of different arrangements and structures. In the embodiments of FIG. 4, support members 450 each comprise a tubular shaped enclosed region 431. Enclosed regions 451 are generally linear, but some, such as regions 450E and 450F, have a curvature that follows the curve of the edge of flaps 121. Enclosed regions 431 are formed within device 400. That is, the walls of enclosed regions 431 are formed from the body of device 400. In certain embodiments, the portions of device 400 forming the walls of enclosed regions 431 are reinforced with an additional material, such as embedded fibers, or have thickness that is greater than other regions of the device.

It would be appreciated that the shapes and locations for support members 450 provided above are merely illustrative and do not limit embodiments of the present invention. For example, in other embodiments, any of members 450 may be omitted from the device and/or other members may be added.

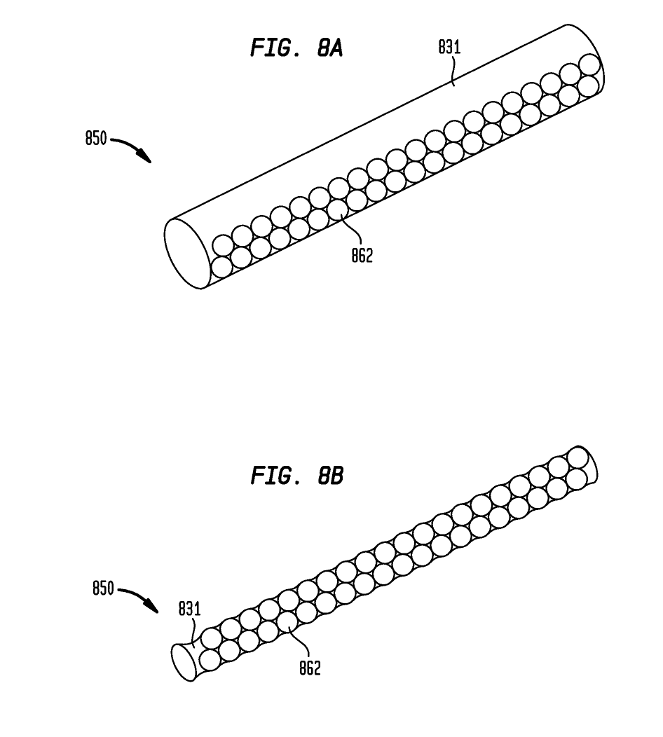

As described elsewhere herein, a transformable support member in accordance with embodiments of the present invention may have a number of different arrangements and structures. In the embodiments of FIG. 4, support members 450 each include an amount of non-compressible powder therein. In use, support members 450 have a first volume in the first, flexible configuration that allows the powder to move freely within enclosed regions 431. However, support members 450 are configured to have a second, smaller volume in the second, rigid configuration. Due to this decrease in volume, the powder is compressed together to form a rigid structure. The volume may be changed by applying vacuum to enclosed regions 431 via a valve or port (not shown) on each support member 450. Further details of such embodiments are provided below with reference to FIGS. 8A and 8B.

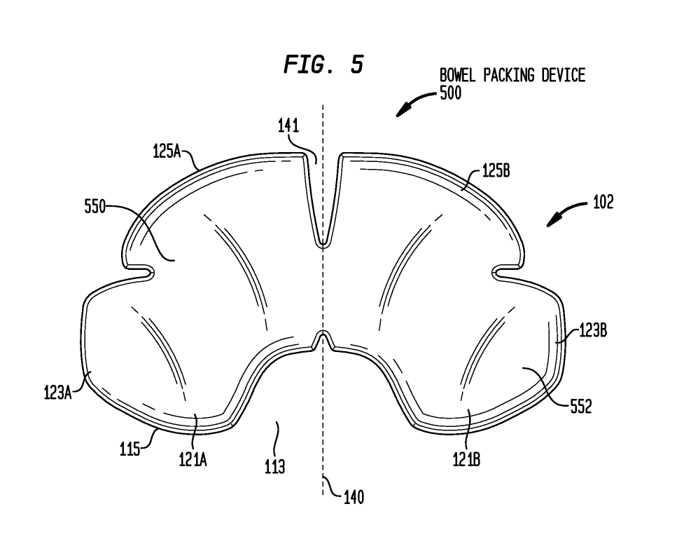

FIG. 5 is a front view of another bowel packing device 500 in accordance with embodiments of the present invention. Device 500 is similar to device 100 of FIG. 1, and includes a central portion 107 and flaps 121, 123 and 125. Organ packing device 500 further comprises a transformable support member 550 disposed in the device. More specifically, support member 550 comprises the substantial entirely of device 500 and as an essentially elliptical shape that is generally symmetrical about a minor axis.

Similar to support member 150 of FIG. 1, support member 550 is configured to have a first substantially compliant configuration and a second substantially rigid or stiff configuration. When support member 550 is in the compliant configuration, central portion 107 and flaps 121, 123 and 125 are flexible and may be compressed or otherwise manipulated by the surgeon. However, when support member 550 are in the rigid configuration, central portion 107 and flaps 121, 123 and 125 are substantially rigid relative to the remainder of the device and/or the subject's tissue.

As described elsewhere herein, a transformable support member in accordance with embodiments of the present invention may have a number of different arrangements and structures. In the embodiments of FIG. 5, support member 550 is substantially similar to support member 150 of FIG. 1 and is configured to be substantially filled with a pressurized fluid via valve or port 552.

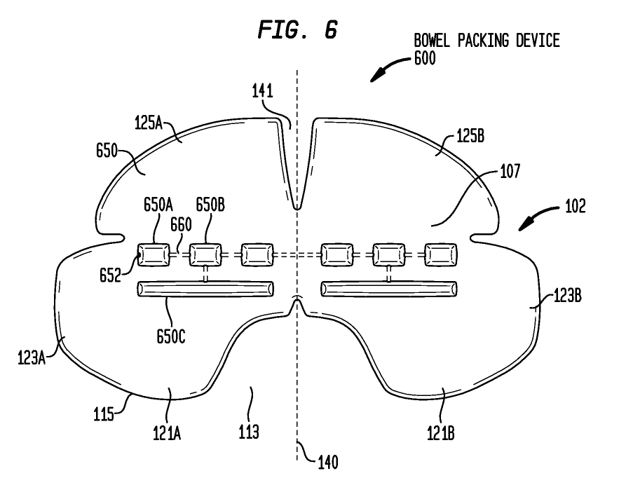

FIG. 6 is a front view of another bowel packing device 600 in accordance with embodiments of the present invention. Device 600 is similar to device 100 of FIG. 1, and includes a central portion 107 and flaps 121, 123 and 125. Bowel packing device 500 further comprises a plurality of transformable support members 550 disposed in central portion 107 of the device. Similar to support member 150 of FIG. 1, support members 650 are configured to have a first substantially compliant configuration and a second substantially rigid or stiff configuration.

As described elsewhere herein, a transformable support member in accordance with embodiments of the present invention may have a number of different arrangements and structures. In the embodiments of FIG. 6, support members 650 are substantially similar to support member 150 of FIG. 1 and are configured to be substantially filled with a pressurized fluid via valve or port 652. As shown, support members 650 are fluidically coupled to one another via fluid passages 600 embedded in device 600.

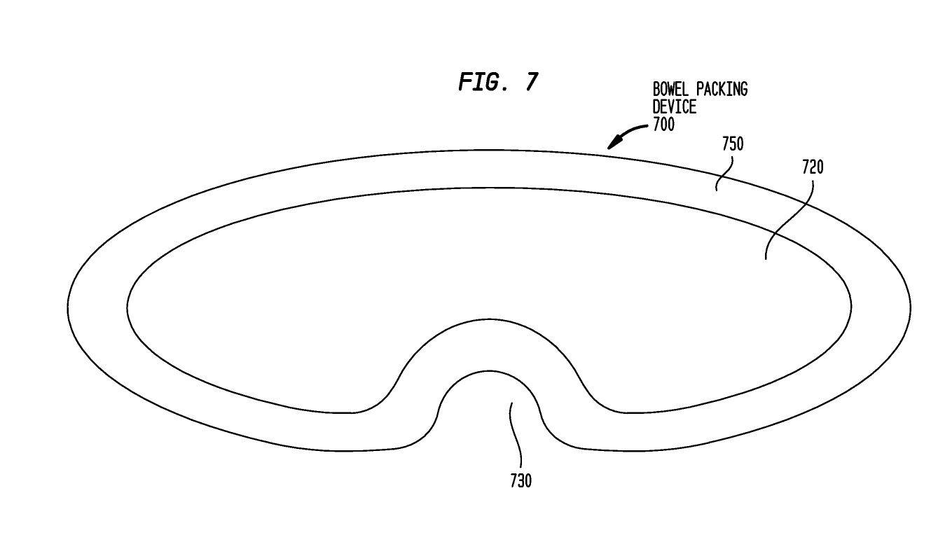

FIG. 7 is a front view of another bowel packing device 700 in accordance with embodiments of the present invention. As shown, device 700 includes a support member 750 in the form of an outer frame that surrounds an interior diaphragm 720. Diaphragm 720 is formed from an elastomeric compound, and may be, in certain embodiments, a plastic film having substantially uniform thickness.

As shown, support member 750 has an essentially elliptical shape, and includes a notch 730 therein. Notch 730 is sized, shaped and located to accommodate a subject's spine when device 700 is inserted into the subject.

Similar to support member 150 of FIG. 1, support member 750 is configured to have a first substantially compliant configuration and a second substantially rigid or stiff configuration. Additionally, support member 750 may be implemented in accordance with any of the embodiments described herein. For example, in certain embodiments, support member 750 may included an enclosed region configured to receive a pressurized fluid, while in other embodiments support member 750 is enclosed region containing a transformable material, such as a wax. In other embodiments, support member may include a powder and be configured to undergo a volume change.

As noted above with reference to FIG. 4, a support member in accordance with certain embodiments of the present invention may comprise an enclosed region containing a powder. FIGS. 8A and 8B are perspective views of an exemplary support member 850 comprising an enclosed region 831. Within enclosed region 831 are spherical powder particles 862. For ease of illustration, only a few, enlarged powder particles are shown. It would be appreciated that in practice the size of powder particles 862 may be substantially smaller and greater of particles may be included.

As noted above, support members in accordance with embodiments of the present invention are configured to have a first substantially compliant configuration and a second substantially rigid or stiff configuration relative to the remainder of the device in which the structure is disposed and/or relative the subject's tissue. FIG. 8A illustrates support member 850 in the first, compliant configuration. As shown, enclosed region 831 has a sufficiently large volume that allows particles 862 to flow within the region. Due to this extra room, support member 850 is substantially conformable by the surgeon.

FIG. 8B illustrates support member 850 in the second, rigid configuration. As shown the volume of enclosed region 831 has been significantly reduced from the volume of FIG. 8A. In these embodiments, particles 862 are substantially non-compressible such that, as the volume is reduced, the particles are positioned abutting one another and are locked together by the outer surfaces of enclosed region, thereby forming a substantially rigid structure. The volume of enclosed region 831 may be reduced by applying vacuum pressure to the region.

FIGS. 8A and 8B illustrate the use of spherical particles 862. It should be appreciated that the spherical shape of particles 862 are merely illustrative, and other shapes may also be used. However, spherical shaped particles have the advantage of facilitating free flow of the particles in the first, complaint configuration.

Additionally, FIGS. 8A and 8B illustrate embodiments in which all of the particles 862 have the same size and shape. It would be appreciated that in alternative embodiments particles of different sizes and shapes may be used within the same support structure. For example, referring to the embodiments of FIGS. 8A and 8B, smaller spherical particles may be added to fill in the spaces between the larger particles 862.

The powder used in embodiments of the present may be formed from, for example, polyesters, polyurethanes, polycarbonates, PVC, inorganic silicates, carbonates, etc. In specific embodiments, the powder is biocompatible and bio-resorbable (polyesters, poly(amino acids), polyanhydrides, polyorthoesters, polyurethanes, polycarbonates, and copolymers of poly(lactic acid) and poly(glycolic acid), copolyesters of ε-caprolactone, trimethylene carbonate, and para-dioxanone) such that, if the support member would rupture, the particles would not damage the patient's tissue.

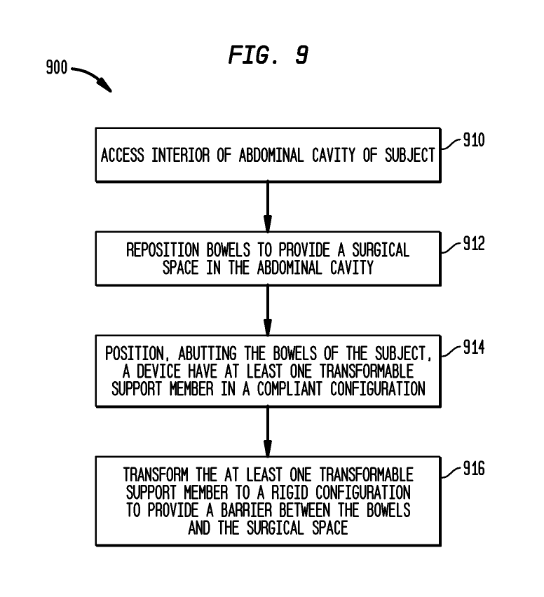

FIG. 9 is a flowchart illustrating an exemplary method 900 for using a organ packing device having at least one transformable support member configured to transform from a first substantially compliant configuration to a second substantially rigid configuration. Method 900 begins at step 910 where a surgeon accesses the interior of an abdominal cavity of the subject. At step 912, the surgeon repositions the organs of the subject to provide a surgical space in the abdominal cavity.

Next, at step 914, while the transformable support member is in the compliant configuration, the surgeon positions the organ packing device abutting the organs of the subject. At step 916, the support member is transformed from the first, compliant configuration to the second rigid configuration to provide a barrier between the surgical space and the subject's organs.

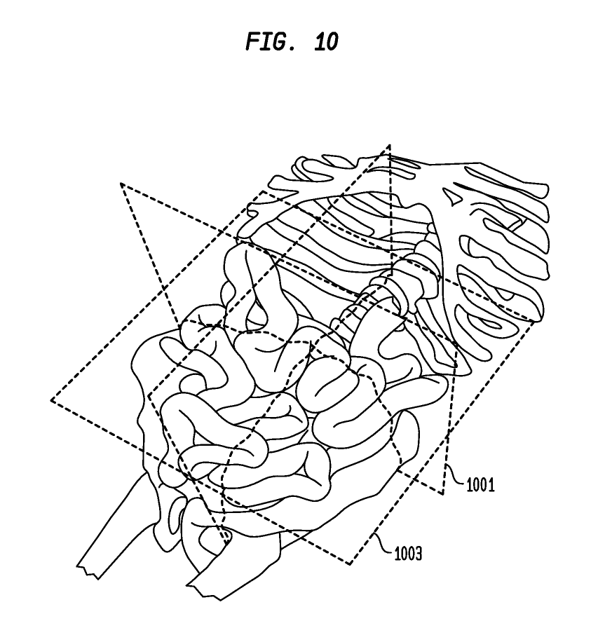

As previously noted, organ packing devices in accordance with embodiments of the present invention are preferably made in different sizes for use in subjects of different sizes (e.g., children and adults). FIG. 10 illustrates relative dimensions of the average human abdomen that were used to determine the dimensions of an exemplary device for use in adult human with the transverse 1001 and coronal 1003 planes indicated. Using the measurements of the adult human abdominal cavity and the devices, the appropriate dimensions for a organ packing device can be determined for use in a subject other than an adult human provided with the dimensions of the abdominal cavity (human child, dog, cat, other mammal). Anthropologic data may be used to determine the small, medium, and large sizes designed to fit at least 95% of the adult human population. This flexibility of the device allows it to conform to cavities that may otherwise be too big or too small. In a embodiment, the small size will be about 5.20 inches total in height and about 8.70 inches total in width; the large size will be about 7.50 inches in height and about 12.50 inches in length; the medium size of the device is about 6.53 inches in total height, including the body and flaps, and about 10.92 inches wide.

The packing devices of the invention can also include other components such as coatings to reduce sticking of the device to the organ by coating with polymers, particularly biocompatible polymers, of with commercially available coatings such as Seprafilm®. The coatings may be drug eluting. The coatings may be applied by bulk application, molecular conjugation with the body material, or through nanostructure formation. Examples of possible coatings include: SEPRAFILM®, INTERCEED®, SIROLIMUS®, PACLITAXEL®, EVEROLIMUS®, TRANILAST®, DACRON®, SPRAYGEL®, ADHffiIT®, TEFLON®, PRECLUDE® Gore, SyntheMed REPEL-CV®, DuraGen, ADCON'M P (Gliatech), REPEUM and RESOLVE™ (Life Medical Sciences), INTERGEL™ (formerly LUBRICOAT®), icodextrin, hyaluronic acid, heparin, dextran, tissue plasminogen activator, corticosteroids, non-steroid inflammatory drugs (NSAIDS) such as ibuprofen, chondroitin sulfate, carboxymethylcellulose, dexamethosane, tissue plasminogen including recombinant tissue plasminogen, oxyphenbutazone, collagen, collagen inhibitors, polylactic acid, polyglycolic acid, alginic acid, polycaprolactone, glycosaminoglycans, polyethylene oxide (PEO), polyethylene oxidepolypropylene oxide copolymer in any monomeric ratio (pEG-PPO-PEG), hydroxy ethyl methyl acrylate (HEMA), poly(N-isopropylacrylamide) (NIPAAm), polytetraflouroethylene (PTFE), polyesters, and silane, or modification by radio frequency gas discharge (RFGD), and radiation grafting, polytetrafluoroethylene (PTFE), polylactic acid, polyglycolic acid, alginic acid, polycaprolactone, glycosaminoglycans, HEMA, ePTFE, polyesters, carboxymethylcellulose, dexamethasone, tissue plasminogen including recombinant tissue plasminogen, oxyphenbutazone, corticosteroids, icodextrin, hyaluronic acid, hyaluronan, and collagen inhibitors.

Alternatively, packing devices can be coated with agents, for example, anti-microbial agents such as anti-viral agents or anti-bacterial agents. The use of such agents may be useful for the protection of the subject as well as the surgical staff and to reduce the possibility of transmission of infection from subjects infected with HIV, hepatitis, especially drug-resistant forms of hepatitis, methicillin resistant staphylococcus aureus (MERSA), etc.

Embodiments of the present invention have been primarily described with reference to transformable support members embedded or disposed in the device. However, in certain embodiments, the support members are not necessarily disposed or embedded in the device, but rather may be disposed on the surface of the device.

Also as noted above, organ packing devices in accordance with embodiments of the present invention may be inserted into a subject via a laparotomy, or via a laparoscopic procedure. In embodiments in which the device is configured for insertion via a laparoscopic procedure, the device is sufficiently collapsible that the barrier may be inserted into an abdomen via a trocar or substantially small incision, the size of which is known in the art. The size of a such an incision is small when compared to the incision typically made through the ventral side of the subject during a laparotomy.

The collapsible device may be collapsed (e.g., rolled, folded or otherwise bunched together) to fit into the cannula of the trocar, etc. Sufficient force applied to the collapsible device causes the device to move through the cannula of the trocar and into the abdominal cavity. Once in the abdominal cavity, the device is uncollapsed or expanded (e.g., unrolled, unfolded, unbunched, etc.) to expand to the configuration(s) detailed herein. The transformation of the support members from the first to second configurations provides the device with sufficient structural rigidity after it is expanded such that it maintains the organs in a retained state.

Once the device is no longer needed in the abdomen, the device may be re-collapsed so that it may be withdrawn from the abdomen through the cannula of the trocar and/or through the incision in the abdomen.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All documents, patents, journal articles and other materials cited in the present application are hereby incorporated by reference.

Reference herein to “one embodiment” or “an embodiment” means that a particular feature, structure, operation, or other characteristic described in connection with the embodiment may be included in at least one implementation of the invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

What is claimed is:

1. An elastomeric device for packing the organs of a subject comprising:
a central portion and one or more flaps collectively manually positionable within the subject to retain the organs or a portion of the organs of the subject in an operational, displaced position and to provide a surgical operational space; and
at least one transformable support member disposed in at least one of the central portion and one tube to guide the device and to transform the device and the flaps configured to transform from a first substantially compliant configuration to a second substantially rigid configuration.

2. The device of claim 1, wherein the central portion and the one or more flaps form an essentially elliptical shape that is generally symmetrical about a minor axis of the device.

3. The device of claim 1, wherein the at least one transformable support member comprises an enclosed region configured to receive and retain a pressurized fluid therein, and wherein the enclosed region contains substantially no fluid in the first configuration, and is substantially filled with the fluid in the second configuration.

4. The device of claim 3, wherein the fluid is a pressurized gas.

5. The device of claim 3, wherein the fluid is a pressurized liquid.

6. The device of claim 1, wherein the at least one transformable support member comprises an enclosed region containing a transformable material that is a liquid in the first configuration and a solid in the second configuration.

7. The device of claim 6, wherein the transformable material is a wax.

8. The device of claim 1, wherein the at least one transformable support member comprises an enclosed region containing a powder, wherein the enclosed region is configured to have a first volume in the first configuration and a second volume in the second configuration, and wherein the second volume is less than the first volume.

9. The device of claim 8, where the enclosed region has an accessible port that allows a surgeon to place vacuum pressure on the enclosed region.

10. The device of claim 1, further comprising:
a cut-out located on the minor axis of the device, wherein the cut-out is dimensioned to accommodate the spine of the subject.

11. The device of claim 1, wherein the at least one transformable support member is disposed in the central portion.

12. The device of claim 1, wherein the at least one transformable support member in at least one of the flaps.

13. The device of claim 1, wherein the at least one transformable support member comprises a plurality of support members.

14. The device of claim 1, wherein the device is sufficiently flexible to bend around the spine of the subject during packing of the organs.

15. The device of claim 1, further comprising a heat transfer device configured to transfer thermal energy to and/or from the device.

16. The device of claim 1, wherein the heat transfer device comprises at least one of an electrical circuit and a fluid circuit disposed on or in the device.

17. A method of packing organs of a subject with a device including a central portion and one or more flaps, wherein the device comprises at least one transformable support member disposed in at least one of the central portion and the flaps configured to transform from a first substantially compliant configuration to a second substantially rigid configuration, the method comprising:
accessing an interior of an abdominal cavity of the subject;
repositioning the organs to provide a surgical space in the abdominal cavity;
positioning the device having the at least one transformable support member in the first configuration abutting the organs; and
transforming the at least one transformable support member to the second configuration to provide a barrier between the organs and the surgical space.

18. The method of claim 17, wherein the at least one transformable support member comprises an enclosed region, and wherein transforming the at least one transformable support member to the second configuration comprises:
substantially filling the enclosed region with a pressurized fluid.

19. The method of claim 18, wherein substantially filling the enclosed region with a pressurized fluid comprises:
substantially filling the enclosed region with a pressurized gas

20. The method of claim 18, wherein substantially filling the enclosed region with a pressurized fluid comprises:
substantially filling the enclosed region with a pressurized liquid.

21. The method of claim 17, wherein the at least one transformable support member comprises an enclosed region containing a transformable material, and wherein transforming the at least one transformable support member to the second configuration comprises:
transforming the transformable material from a liquid to a solid.

22. The method of claim 21, wherein transforming the transformable material from a liquid to a solid comprises:
applying thermal energy to the transformable material.

23. The method of claim 17, wherein the at least one transformable support member comprises an enclosed region containing a powder, and wherein transforming the at least one transformable support member to the second configuration comprises:
placing a vacuum on the enclosed region to reduce the volume of the enclosed region.
